(19) United States
(12) Patent Application Publication
(10) Pub. No.: US 2005/0027162 A1
(43) Pub. Date: Feb. 3, 2005

(54) Human doll and method therefor

(76) Inventor: Steve Paled, Palmdale, CA (US)

Correspondence Address: WEISS & MOY PC, 4204 NORTH BROWN AVENUE, SCOTTSDALE, AZ, 85251, US

(21) Appl. No.: 10/631,416

(22) Filed: Jul. 30, 2003

(51) Int. Cl. A61F 5/00; B29C 33/40; H04B 3/00

(52) U.S. Cl. 600/38; 264/222; 381/77

(57) ABSTRACT

A life-size human doll is preferably formed from a composite of silicone and polyurethane. Silicone may be utilized in sensitive regions, including the face, genital area, anal area, and breast area. It is preferred that the human doll be anatomically correct. It is further preferred to provide the human doll with a heart beat simulator, and with a high quality sound production system capable of producing voice-type sounds. The sounds are preferably downloadable from a network, in a plurality of languages.

### FIELD OF THE INVENTION

This invention relates generally to dolls and, more particularly, a human-size doll having features intended to confer lifelike qualities.

### BACKGROUND OF THE INVENTION

Human size dolls have become increasingly sophisticated in recent years, when it comes to endowing them with life-like qualities. Current models are known to have internal skeletons, moveable joints, hair, and a silicone skin.

However, there are certain drawbacks associated with prior art human dolls. The use of silicone as a skin material imparts a life-like quality, but results in a doll that is relatively heavy. For example, a typical silicone skin doll can weight more than 100 pounds. Such weight can make it difficult for an individual to move the human doll from one place to another, or to manipulate the positioning of the doll within a single location.

In addition, prior art human dolls do not produce better-than CD-quality voice-type sound. More particularly, high quality sound that is identifiable with a particular human, after whom the doll is modeled. Still further, prior art dolls do not provide a mechanism for downloading sounds from the Internet or the like, to be played by the doll. Prior art dolls lack in other areas as well, when it comes to qualities that produce a lifelike appearance. These include the generation of a heart beat.

The present invention satisfies these needs, and provides other, related, advantages.

### SUMMARY OF THE INVENTION

It is an object of the present invention to provide a human doll comprising, in combination: a life-sized body including a head, face, genital area, anal area, and chest region; and high quality sound production means located in an interior portion of the life-sized body, and adapted to play a recording of a human voice.

It is a further object of the present invention to provide a human doll comprising, in combination: a life-sized body including a head, face, genital area, anal area, and chest region; and a heart beat simulator located proximate the chest region.

It is a still further object of the present invention to provide a method for manufacturing a anatomically correct doll replica of a live human subject comprising the steps of: laser scanning a live human subject; and casting the live human subject.

### BRIEF DESCRIPTION OF THE DRAWINGS

FIG. 1 is a front view of a female human doll, consistent with an embodiment of the present invention.

FIG. 2 is a back view of the female human doll of FIG. 1.

FIG. 3 is a front view of the head portion of the female human doll of FIG. 1.

FIG. 4 is a front view of a male human doll, consistent with an embodiment of the present invention.

FIG. 5 is a back view of the male human doll of FIG. 4.

FIG. 6 is a perspective view of a female human doll, in a preferred orientation.

FIG. 7 is a side, cross-sectional view, of the genitals portion of a male human doll consistent with an embodiment of the present invention.

FIG. 8 is a side, cross-sectional view, of the mouth portion of a human doll consistent with an embodiment of the present invention.

FIG. 9 is a perspective view of a control panel portion of a human doll consistent with an embodiment of the present invention.

### DETAILED DESCRIPTION OF THE PREFERRED EMBODIMENTS

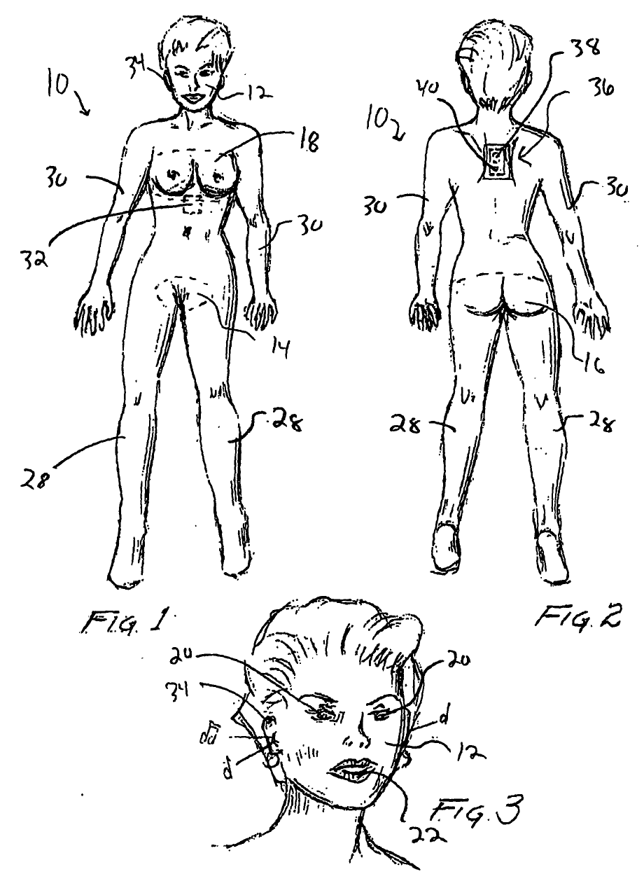

Referring first FIGS. 1-2 and 6, an embodiment of a female human doll 10 (referred to as “female doll 10”) consistent with the present invention is shown.

It should be noted that the female doll 10 should be substantially the size of an adult female. It is preferred that the female doll 10 be patterned after the face and body of a publicly known woman—for example, a film star, model, or the like. As will be described more fully below, it is preferred that the process of manufacturing the female doll 10 include both a laser scanning and life casting of a live subject, to ensure anatomical correctness. In this regard, a scan tends not to pick up pores and wrinkles in an effective way, while casting will capture these. On the other hand, a scan is more effective than body casting at capturing the body's musculature.

There are several alternatives when it comes to the surface material for the female doll 10. Prior art dolls are known to provide a silicone covering for essentially the entire human doll. An advantage associated with silicone is that it is relatively life-like. Disadvantages include that it is relatively heavy. It is also cold to the touch, a quality that can require the warming of the human doll in a bath prior to handling.

Preferably, instead of covering the entire female doll 10 with silicone, it will be preferred to utilize a polyurethane material for most of the body surfaces. Self-skinning polyurethane is lighter, warmer (it generally maintains room temperature), and less expensive than silicone and more durable than non-skinned polyurethane. On the other hand, because of its superior life-like qualities, it will be desired to provide silicone over certain of the body surfaces. These include one or, preferably all, of the following: the face 12, vaginal area 14, anal area 16, and breast area 18. With particular regard to the breast area 18, in order to provide a more life-like feel, it is preferred to provide a dual-layer of silicone, consisting of a firm outer surface and a gel-type, interior layer. With particular regard to the face 12, it will be desired to provide a silicone insert having the front and sides of the face, and to couple that insert to the head of the female doll 10.

With respect to those body portions containing orifices, specifically the vaginal area 14, the anal area 16, and the mouth 22, it will be desired to provide an orifice having a life-like feel and configuration. The surface of such orifices should be lined with silicone. It is further desired to provide a vibrator in the region of the orifice in the vaginal area 14, to increase the life-like sensation for a person interacting with the female doll 12 via such orifice. It is further preferred to provide a vibrator having variable speed and/or intensity.

Referring briefly to FIG. 8, additional detail is provided of a preferred embodiment of the mouth 22 and oral cavity region 24. It is preferred to line the superior and inferior surfaces of the oral cavity region 24 with a series of pliant ridges 26, formed from silicone. These should be dimensioned so that they will form a relatively airtight seal with a male sexual organ inserted therein. Such an effect will cause a relatively pleasurable suction sensation when the male sexual organ is removed from the oral cavity region 24.

Referring in more detail to the face 12, it will be preferred to provide eyes 20 that are comprised of glass. Still further, it will be preferred to match the color of the eyes 20 to that of the live subject of the female doll 10.

Referring now to FIG. 6, it is noted that where polyurethane is utilized to cover a majority of body surfaces, including particularly the legs 28, the polyurethane-coated surfaces are less flexible than silicone coated ones. As one consequence of this, and in the absence of an internal joint system, it is generally not readily possible to re-position such areas. As a result, it will be desired during the manufacturing process to select a fixed, preferred posture for the female doll 10.

The posture shown in FIG. 6, with knees and hands on the ground and the legs bent about the knees, is preferred where re-positioning of the legs (and perhaps also arms) is not possible. This posture along with spread knees permits the satisfaction of a plurality of preferred positions of copulation. In one embodiment, the arms 30 are flexibly attached via an interior joint (not shown) in the shoulder region. Such an arrangement would permit the arms to be positioned as shown in FIG. 6, as shown in FIG. 1, and in an above the head orientation (not shown). Where the arms are flexibly attached on the female doll 10, it will only be necessary to pre-position, during manufacturing, the legs 28 in the manner shown in FIG. 6 or described above.

Referring now to FIG. 1, the box in the chest region signifies a heart beat simulator 32. The heart beat simulator 32 emits a beating sound that is intended to simulate a human heart beat. It is desired that the heart beat simulator 32 be activated when the female doll 32 senses movement, as when a person interacts with it. (Alternatively, the heart beat simulator 32 could be turned on and off with a person activated control, as discussed below.) The beating sound should be discernible when an ear is positioned proximate the heart beat simulator 32, but should not otherwise be audible.

It is further preferred to provide some form of sound production means internal to the female doll 10, preferably in the region between the shoulderblades. Preferably, the sound production means is an MP3 player, or other means capable of storing and supplying higher than CD-quality sound, so as to be more life-like. (The term “high quality sound” as used herein means higher than CD-quality sound.) Alternatively, the sound production means may be an audiocassette player or compact disc player. The sound production means is coupled to speakers, which are preferably located so as to emit sound from the ears 34. See FIG. 3. There are preferably at least two speakers, so that sound production has a stereo or surround-sound quality.

The sounds emitted from the sound production means are preferably words that will have the appearance of being spoken by the female doll 10, in as life-like a fashion as possible. The words should include, preferably, the types of words that a person might say during sexual intercourse or other intimate activity. It is further preferred that the words be pre-recorded by the live subject of the female doll 10, and that they be stored in a format which allows generation of a high quality sound reproduction of the words. It is further preferred that the voice recording be converted to an MP3 format file. In this capacity, commercial software may be used to process a voice recording by filtering noise and manipulating the recording bit rate. The processed voice recording may then be converted to an MP3 format without distinguishable loss of fidelity using commercial software designed for voice-to-MP3 conversion or by custom software techniques. Still further, it is preferred that the sound production means have the capacity to play words in more than one language. Where there are languages in which the live subject is not able to make a recording, it would be preferred to provide a recording that simulates or mimics the voice of the live subject.

Referring now to FIGS. 2 and 9, it is preferred to provide a control panel 36 proximate the sound production means or at some other desired location on the body of the female doll 10. The control panel 36 may include an on/off switch 38 and a dataport 40, closeable with cover 42. (The cover 42 may be secured with opposing hook and loop means 44, or as otherwise desired.) The on/off switch 38, where provided, activates the sound production means. (Alternatively, the sound production means may be activatable via remote control, as discussed in more detail below, or by motion, as with the hear beat simulator 32.) The dataport 40 permits the uploading of sounds to the sound production means, to be played thereon. (Alternatively, the uploading of sounds could be accomplished via a wireless connection, so as to eliminate the need for a dataport 40.) In this regard, it may be desired to store sounds to a network-accessible location such as an internet server and to provide a purchaser of the female doll 10 with identifying information, such as a username and password, which may be provided upon logging on to a web-site. After log-on, the purchaser may be presented with the option of downloading sounds from a plurality of sound choices. Preferably, the person may choose from different messages, different languages, different voices, etc. It may further be desired to automate the uploading to transfer sounds from the internet server directly to the doll without requiring the intermediate step of first saving the file to a connecting computer.

It should be noted that the control panel 36 could also be utilized to provide access to the interior of the female doll 10 for purposes of inserting batteries. Still further, where re-chargeable batteries are utilized to power the various features of the female doll 10, it would be possible to permit re-charging via a cord plugged into a suitable receptacle on the control panel 36.

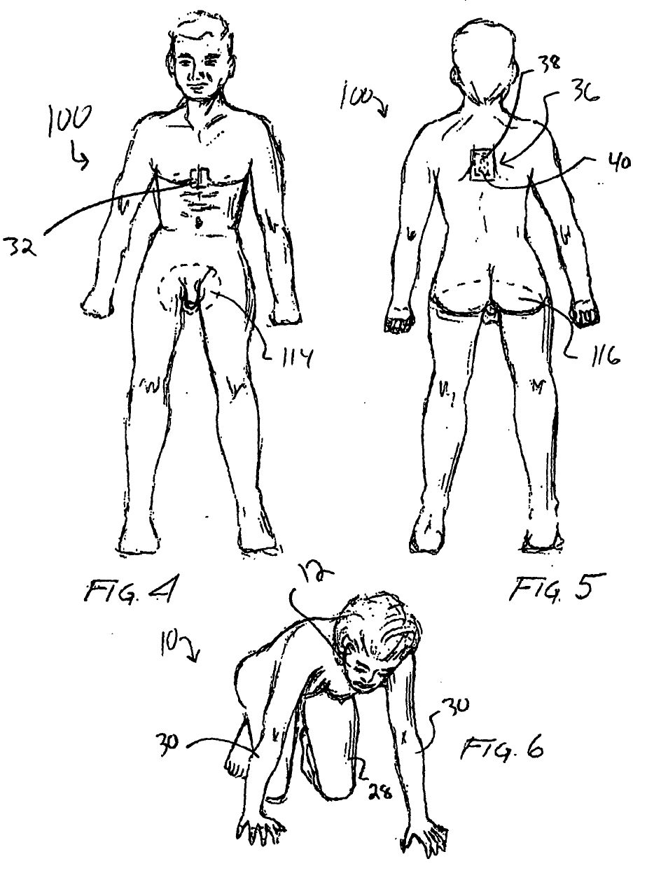

Referring now to FIGS. 4-5, an embodiment of a male human doll 100 (hereinafter “male doll 100”) is shown. As discussed above with respect to the female doll 10, and as indicated with dashed lines, it is desired to provide a male doll 100 that is a composite of lightweight polyurethane and heavier, more life-like silicone. Specifically, with respect to the male doll 100, it is preferred to provide a face 112, genital area 114, and anal area 116 covered with silicone. The use of silicone should include not just external surface areas, but internal orifice linings.

It should be noted that in contrast to the preferred posture of the female doll 10 as shown in FIG. 6, the preferred posture for the male doll 100 is that shown in FIGS. 4-5—i.e., a standing position. Like the female doll 10, it is desired to provide the male doll 100 with a heart beat simulator 32 and sound production means. It may further be desired, as shown in FIG. 5, to provide the male doll 100 with a control panel 36, possibly including an on/off switch 38 and a dataport 40, as described above.

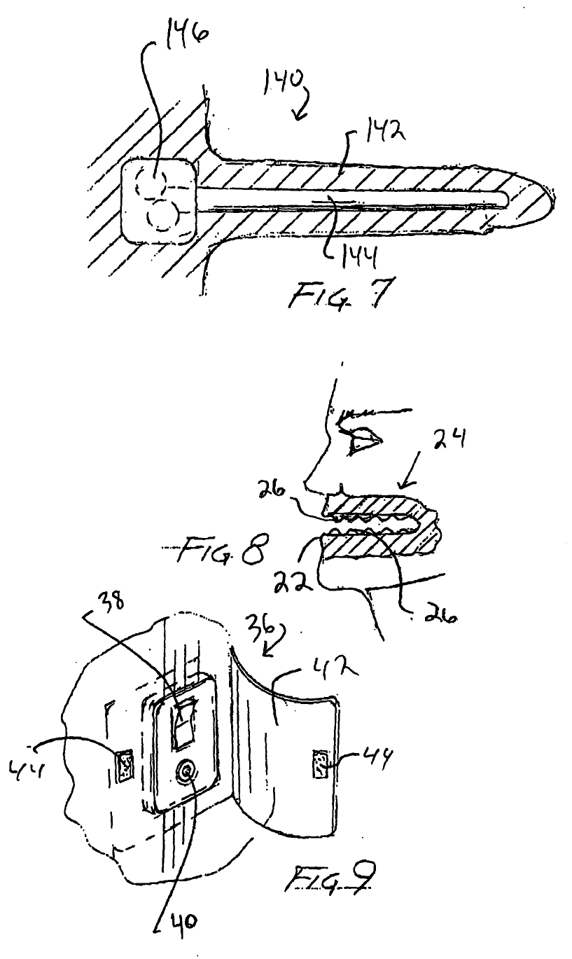

Referring now to FIG. 7, attention is directed to a preferred structure for a penis 140 located on the male doll 100. The main components of the penis 140 include a layer of silicone 142, having a rigid member 144 embedded axially therein. The rigid member 144 may be coupled at its base to a vibrator 146, which vibrator 146 imparts vibrational movement to the rigid member 144.

An advantage of this configuration is that vibration intensity will decrease from the base of the penis 140 to its head. This will result in greater stimulation for a person interacting with the penis 140, the deeper the penetration of the penis 140 into such a person.

It should be noted that for all of the vibrators discussed herein, it is preferred that they be variable with respect to speed. It is further preferred that they be operated via remote control. The remote control is preferably wireless, though a control coupled with wires to the female doll 10 or male doll 100 could also be utilized.

Attention is directed now to a discussion of the assembly of the female doll 10 and male doll 100. While various changes may be made to the manufacturing processes herein described, they are provided as exemplary of preferred embodiments of the assembly process.

In one embodiment, a female doll 10 can be assembled through the following process:
1. Laser scan live subject, to include full nude body, head, hands and feet. Multiple scans should be performed in a series of potentially appropriate positions, to the extent that the finished female doll 10 will exhibit flexibility.
2. Review computerized scan data and determine desired pose or poses.
3. Composite poses if necessary and digitally clean data.
4. Using X-Y axis rigid foam milling machine and render scan data as a 3D rigid foam piece, to include as much detail as is reasonably possible.
5. If the rendering of the data results in multiple pieces, the pieces should be joined together using polyester resin, to achieve homogeneous figure.
6. Determine where the separation of the arms from the body will be and cut rigid foam figure appropriately.
7. Using geometry of foam figure, determine placement and parameters of the face insert, breast, genitalia and anus insert or inserts, vibrator wire way and cavity, MP3 wire way and cavity.
8. Using acquired geometric information, life cast (alginate and plaster bandage) head, breasts, genitalia and anus. Additionally, life cast hands in appropriate positions.
9. Generate clay or hydrocal positives from alginate molds. Sculpt positives as necessary to recreate original detail.
10. Develop, through sculpting and molding, cavities in the rigid foam body, to accept face, breasts, genitalia, and anus inserts, as well as vibrator and MP3 cavities. Design cavities to provide a mechanical body between finished body and inserts.
11. Develop socketed, multi-positionable attachment assembly for arm/body connection.
12. Attach hand positives to the arms.
13. Hardcoat (using polyester gelcoat) foam body and arms.
14. Bodyshop (sand and patch) body and arms as necessary to achieve desired aesthete.
15. Reverse engineer life cast positives to fit into appropriate cavities in the body.
16. Create void on the rear side of breasts positive to accommodate ‘heartbeat’ module.
17. Carve receptacles into arms and body to accept attachment assemblies. Create paths for wire ways to accommodate electronics.
18. Mold body, arms, and hands using appropriate epoxy tooling system.
19. Have self-skinning soft foam positives run from epoxy molds.
20. Resculpt inserts to fit foam body (as original dimensions may have changed as a result of expansion or contraction).
21. Have pubic wig made and acquire head wig. Have both styled appropriately.
22. Mold inserts to accommodate either silicone or thermoplastic injection.
23. Cast parts from insert molds.
24. Insert vibrator and MP3 elements into foam body.
25. Insert heartbeat module into breasts insert.
26. Paint inserts appropriately.
27. Insert face, genitalia, and anus pieces into body.
28. Install wig and pubic hair where appropriate.
29. Test electronics.
30. Test fit arms.
   It should be recognized that the order of these processing steps is, in large part, variable. It should further be recognized that it may be permissible to eliminate one or more of these steps without materially impacting on the quality of the female doll 10 produced.

In another embodiment, the female doll 10 can be assembled through a more simplified process, as follows:
1. The body, excluding the head and genital areas, of the live subject is laser scanned.
2. The head and genital area of the live subject is casted.
3. A foam body corresponding to that of the scanned live subject is generated.
4. A depression is created in the foam body to accept a silicone genital insert.
5. The foam body is hard-coated with polyurethane, and the head is attached.
6. The body is molded.
7. A hero poly body is run.
8. Snaps are obtained for positives of body depressions.
9. Sculpt insert to fit hero body.
10. Mold crotch insert with a core for the vagina.
11. Cast the insert in silicone. Prepaint molds and pre-tint the silicone.
12. Insert the eyes into the head.
13. Attach wig and crotch hair.
14. Paint face, finger and toe nails.
   It should be recognized that, in this embodiment, the female doll 10 features only a single silicone insert—for the genital area 14.

The male doll 100 can be assembled through the following process:
1. Laser scan live subject, to include full nude body, head, hands and feet. Multiple scans should be performed in a series of potentially appropriate positions, to the extent that the finished female doll 10 will exhibit flexibility.
2. Review computerized scan data and determine desired pose or poses.
3. Composite poses if necessary and digitally clean data.
4. Using X-Y axis rigid foam milling machine and render scan data as a 3D rigid foam piece, to include as much detail as is reasonably possible.
5. If the rendering of the data results in multiple pieces, the pieces should be joined together using polyester resin, to achieve homogeneous figure.
6. Determine where the separation of the arms from the body will be and cut rigid foam figure appropriately.
7. Using geometry of foam figure, determine placement and parameters of the face insert, breast, genitalia and anus insert or inserts, vibrator wire way and cavity, MP3 wire way and cavity.
8. Using acquired geometric information, life cast (alginate and plaster bandage) head, breasts, genitalia and anus. Additionally, life cast hands in appropriate positions.
9. Generate clay or hydrocal positives from alginate molds. Sculpt positives as necessary to recreate original detail.
10. Develop, through sculpting and molding, cavities in the rigid foam body, to accept face, genitalia, and anus inserts, as well as vibrator and MP3 cavities. Design cavities to provide a mechanical body between finished body and inserts.
11. Develop socketed, multi-positionable attachment assembly for arm/body connection.
12. Attach hand positives to the arms.
13. Hardcoat (using polyester gelcoat) foam body and arms.
14. Bodyshop (sand and patch) body and arms as necessary to achieve desired aesthete.
15. Reverse engineer life cast positives to fit into appropriate cavities in the body.
16. Carve receptacles into arms and body to accept attachment assemblies. Create paths for wire ways to accommodate electronics.
17. Mold body, arms, and hands using appropriate epoxy tooling system.
18. Have self-skinning soft foam positives run from epoxy molds.
19. Resculpt inserts to fit foam body (as original dimensions may have changed as a result of expansion or contraction).
20. Have pubic wig made and acquire head wig. Have both styled appropriately.
21. Mold inserts to accommodate either silicone or thermoplastic injection.
22. Cast parts from insert molds.
23. Insert vibrator and MP3 elements into foam body.
24. Paint inserts appropriately.
25. Insert face, genitalia, and anus pieces into body.
26. Install wig and pubic hair where appropriate.
27. Test electronics.
28. Test fit arms.

While the invention has been particularly shown and described with reference to preferred embodiments thereof, it will be understood by those skilled in the art that the foregoing and other changes in form and details may be made therein without departing from the spirit and scope of the invention.

For example, it may be desired to provide sound production means as herein described in a female doll 10 or male doll 100 having a full silicone covering. It may be desired to provide a heart beat simulator 32 in a female doll 10 or male doll 100 having a full silicone covering.

1. A human doll comprising, in combination:
a life-sized body including a head, face, genital area, anal area, and chest region; and
high quality sound production means located in an interior portion of said life-sized body, and adapted to play a recording of a human voice.

2. The human doll of claim 1 wherein said life-sized body is anatomically correct and wherein said high quality sound production means plays a recording of a voice substantially indistinguishable from an actual human voice.

3. The human doll of claim 1, wherein said life-sized body has a likeness of a selected human model, said life-sized body is an anatomically correct duplicate of the body of said selected human model, said high quality sound production means plays a recording of a voice of said selected human model substantially indistinguishable from said actual voice of said selected human model, whereby a copulative act with said human doll provides a visual, aural, and tactile sensory experience substantially similar to a visual, aural, and tactile sensory experience provided by said copulative act with said selected human model.

4. The human doll of claim 1 wherein said life-sized body has been manufactured from a laser scan and a cast of a live subject.

5. The human doll of claim 1 wherein said life-sized body is a composite of polyurethane and silicone.

6. The human doll of claim 5 wherein said life-size body has a silicone covering for at least one of said face, said genital area, said anal area, and said chest region.

7. The human doll of claim 1 wherein superior and inferior surfaces of an oral cavity region within said face is lined with a series of pliant ridges formed from silicone, dimensioned to form a relatively airtight seal with a male sexual organ inserted therein.

8. The human doll of claim 1 wherein said face has eyes located therein that are comprised of glass.

9. The human doll of claim 8 wherein a color of said eyes matches a color of eyes of a live subject of said human doll.

10. The human doll of claim 1 wherein said human doll is fixed into a preferred posture during manufacturing, said preferred posture having legs of said human doll forming a first acute angle with hips of said human doll, said legs having thighs extended anteriorly and superiorly and having knees spread to accommodate the positioning of a person therebetween, and said legs being bent about said knees at a second acute angle substantially similar to said first acute angle.

11. The human doll of claim 10 wherein arms of said human doll are fixed so that they extend substantially directly forward, so that said human doll may be placed on the ground in a hands and knees posture.

12. The human doll of claim 1 wherein said recording consists of words a person might say during sexual intercourse or other intimate activity.

13. The human doll of claim 12 wherein said words are recorded by a live human subject of said human doll.

14. The human doll of claim 13 wherein said high quality sound production means is adapted to alternately play said words in one of a plurality of languages.

15. The human doll of claim 1 wherein said recording is downloaded to said high quality sound production means from a network.

16. The human doll of claim 1 wherein said sound production means is coupled to at least two speakers.

17. The human doll of claim 16 wherein said speakers are located proximate ears located on said head of said life-sized body, and are configured to broadcast sound through said openings in said ears.

18. The human doll of claim 1 wherein said sound production means is an MP3 player.

19. The human doll of claim 1 wherein said human doll is male and wherein said male human doll has a penis comprising:
a layer of silicone; and
a rigid member located below said layer of silicone;
wherein said rigid member is coupled at its base to a vibrator, which vibrator imparts vibrational movement to said rigid member;
wherein vibration intensity decreases from a base of said rigid member to a head of said rigid member.

20. The human doll of claim 2, further comprising a heart beat simulator located proximate said chest region.

21. The human doll of claim 20 wherein said heart beat simulator is activated by movement of said life-size body.

22. A method for manufacturing a human doll comprising the steps of:
laser scanning a live human subject; and
casting said live human subject.

23. The method of claim 22 wherein said step of laser scanning includes scanning a full nude body, head, hands and feet of said live human subject.

24. The method of claim 23 wherein said step of casting said live human subject includes a casting of a head, breasts, genitalia, anus and hands of said live human subject.

25. The method of claim 22 further comprising the steps of:
scanning a full nude body, head, hands and feet of said live human subject;
reviewing computerized scan data;
determining desired pose or poses for said human doll;
digitally cleaning said scan data;
using X-Y axis rigid foam milling machine to render scan data as a 3D rigid foam piece;
determining where separation of arms from the body will be and cut rigid foam figure appropriately;
using geometry of said foam figure, determining placement and parameters of the at least one of face, breast, genitalia and anus inserts;
life casting with alginate and plaster bandage head, breasts, genitalia and anus of said live subject;
generating one of clay and hydrocal positives from casting molds;
sculpting positives as necessary to recreate original detail;
developing cavities in said rigid foam body, to accept said face, genitalia, and anus inserts, as well as vibrator and MP3 cavities;
developing socketed, multi-positionable attachment assembly for arm/body connection;
attaching hand positives to said arms;
hardcoating (using polyester gelcoat) said rigid foam body and said arms;
sanding and patching said rigid foam body and said arms as necessary to achieve desired aesthete;
reverse engineering life cast positives to fit into appropriate cavities in said rigid foam body;
creating receptacles in said arms and rigid foam body to accept attachment assemblies;
molding said rigid foam body, said arms, and said hands using appropriate epoxy tooling system;
running self-skinning soft foam positives from epoxy molds;
providing pubic wig and head wig;
molding said inserts to accommodate either silicone or thermoplastic injection;
casting parts from insert molds;
inserting vibrator and high quality sound production means into foam body;
painting said inserts appropriately;
inserting face, genitalia, and anus pieces into said rigid foam body; and
installing said head wig and said pubic wig.

26. A method of providing a high-quality voice recording to a human doll comprising, in combination, the steps of:
recording a human voice;
converting said recording into an MP3 format file; and
transmitting said MP3 format file to a human doll to permit said human doll to provide a human voice.

27. The method of claim 26 further comprising:
storing said MP3 format file at a network-accessible location;
coupling a sound production means of said human doll to a computer for receiving said MP3 format file; and
connecting said computer to said network for receiving said MP3 format file from said network and for sending said MP3 format file to said sound production means of said human doll.

28. The method of claim 27, wherein said transmitting is performed without a need for an intermediate step of first storing said MP3 format file to a permanent storage device of said computer before forwarding said MP3 format file to said sound production means of said human doll.
